(19) United States
(12) Patent Application Publication
(10) Pub. No.: US 2006/0013862 A1
(43) Pub. Date: Jan. 19, 2006

(54) Onychomycosis: a new process for cure

(76) Inventor: Jerry Martin Held, Monterey, CA (US)

Correspondence Address: Jerry M. Held, P O Box 1342, Monterey, CA, 93942, US

(21) Appl. No.: 10/894,070

(22) Filed: Jul. 19, 2004

(51) Int. Cl. A61K 9/70

(52) U.S. Cl. 424/443

(57) ABSTRACT

A new process and design, applicable to essentially all patients with onychomycosis, that represents a major ‘low-tech’ breakthrough in curing fungal nail infections that are a major source of irritation, morbidity, expense and mortality. The process begins with removal of the overlying infected nail (hands or feet). This achieves a primary ‘debulking’ of the infection, and subsequently allows access to the underlying common infection of the nail plate/bed and vascular supply. Removal can be done painlessly, inexpensively and safely in one of two manners, as detailed in the ‘SUMMARY OF THE INVENTION.’Following removal of the overlying infected nail, the underlying nail bed is systematically treated for 4 to 7 days to eliminate remaining fungal and bacterial infection. The nail bed, once or twice daily, is first soaked in a warm hypertonic solution of Epsom salts, sea salts and vinegar for 1 to 5 minutes. The nail bed is then dried vigorously and a mixture of antibacterial and antifungal cream or spray is then applied. This area is then covered until the subsequent treatment cycle of soaking, drying and antibiotic application is repeated.

Mycotic and bacterial cure is achieved at the end of one week. As with any available treatment, new nail begins to form in 4 to 6 weeks, with complete new, uninfected nail present at 3 to 4 months.

### BACKGROUND OF THE INVENTION/PROCESS

The present process relates to a novel approach to curing infections of nails. This process is a ‘low-tech’ approach that is safe, inexpensive, accessible to all patients and extremely effective. It therefore strongly differs from currently available treatments, which tend to be very expensive, largely ineffective, of significant danger to many patients, and largely unavailable due to cost and risk. “Fungal infections of the nails are a major health concern worldwide (see Infectious Disease Clinics of North America-17-2003-87-112D). It has been estimated that up to 20% of the population in the United States has a dermatophytic infection.” . . . “It accounts for up to 50% of all nail disorders. . . , . . . Incidence in adults over 70 years of age may be as high as 40% because of numerous factors, such as age-related decrease in immune function, lower vascular efficiency, and less efficient T cells and phagocytes.”

“Up to a third of diabetic patients may develop onychomycosis . . . They also experience a higher rate of complication, with disease spreading more rapidly to other nails, or resulting in the development of osteomyelitis and cellulitis, which may progress to necrosis and result in amputation if not treated aggressively and appropriately . . . Whereas some infections are merely unsightly or annoying, others may cause significant morbidity and mortality, especially in the elderly and those with significant illness or immune compromise.”

“Some forms of dermatomycosis are increasing in incidence. Although newer oral antifungals have significantly affected the efficacy and rapidity of treatment of many fungal infections, side effects, drug interactions, and resistant organisms have created a challenge to find safer and more effective treatments . . . Some are quite expensive, have side affects and organ toxicity that may or may not be tolerable, and have significant drug interactions . . . Hepatotoxicity is a concern, especially with long-term use as in the treatment of onychomycosis. Other common side effects include rash, headache, nausea and vomiting, and photosensitivity. Less common ones include arthralgia, peripheral neuritis, memory lapse, confusion, and insomnia.”

“Less that 20% of cases of tinea unguium respond to topical treatment, because of poor penetration to the nail bed. Cicolopirox 8% nail lacquer may be effective in mild to moderate (early) onychomycosis . . . Treatment may take 6 months to one year to be effective, with a cure rate of approximately 5 to 8%.”

“ . . . Mycological cure without second intervention treatment was found in 34 (46%) of the 74 terbinafine-treated subjects and 10(13%) of the 77 itraconazole-treated subjects. Mycological and clinical relapse rates were significantly higher in itraconazole vs terbinafine-treated patients (53% vs 23% and 48% vs 21% respectively.)” (3:Arch. Dermatol. 2002 March;138 (3) 353-7).

The presented invention would be a notable advancement in the field. We live at a time where obesity and obesity related medical problems such as diabetes have become one of our major national priorities. Onychomycosis is one problem threatening obese diabetic patients that has until now been almost impossible to successfully eradicate. Impossible because the expense and risk of currently available treatments is too great. Unfortunately medical expense threatens our country as much as does obesity and diabetes.

This invention would allow cure of a significant medical disease without morbidity, mortality or significant expense. Discomfort is minimal as described below. Almost painless in the hands of an experienced practitioner. Toxicity approaches zero. No monitoring of liver and cell counts is necessary. Physician visits are minimal. Cost should be less than one-hundred dollars, in contrast to costs ranging from one-thousand to four thousand dollars with established treatments. Initial anecdotal reports by this physician are of very high cure rates and far superior patient compliance and preference. Trials with definitive data are pending in more conclusive, long-term studies.

### SUMMARY OF THE INVENTION

The inventor of the above process is a physician practicing chiefly among poor, underinsured farm workers. The incidence of onychomycosis in this population is estimated at 85%, while the prevalence of insurance and financial resources less than 50%. Very few of these patients can afford oral treatment regimens, and most are contraindicated by the high incidence of multiple medications and comorbid conditions present in this population. Compliance continues to be a significant problem with treatments requiring long-term daily care, repeated laboratory tests and physician visits. Dr. Held's process/invention represents a common sense, ‘low-tech’ solution to a world-wide problem that is a major source of morbidity, mortality and cost. This process, elegant in its simplicity, would be available to all patients and require only a simple, straightforward training of health care professionals and/or patients. Removal of the overlying infected nail can be done in one of two manners.

In one process, a ‘digital’ block of lidocaine or a similar anesthetic agent is administered. Then the area between the overlying nail and underlying nail bed is painlessly and safely separated by blunt dissection using a tweezers, hemostat or similar device. Two to three days later, the overlying infected nail (now the consistency of paper) is removed by cutting with a scissors. A thin strip of overlying nail is left against the skin (cuticle area.) The exposed nail bed is then conveniently covered with the antibiotic cream and then with a bandage, gauze, tape or ‘finger’ from a medical glove.

In a second process, the overlying infected nail is removed gradually by piecemeal exposure of the space between the overlying nail and underlying plate. This is done by first soaking the digit in a solution of Epsom salt in warm water, or Epsom salts with various composition of sea salts and vinegar. Following 3 to 5 minute soaking, an antibiotic cream (ideally a mixture of antibacterial cream or ointment and common antifungal cream or ointment) is pressed comfortably into the exposed space between the overlying nail and underlying plate. This process is repeated daily until the antibiotic cream has been pressed into the entire potential space between the overlying nail and underlying plate. At this point, as with the first process outlined above, the overlying infected nail had the consistency of paper and can be removed easily with a scissors.

The invention/process possesses other objects and advantages, especially as concerns particular characteristics and features thereof which will become apparent as review and specification continues.

### BRIEF DESCRIPTION OF THE VIEWS OF THE DRAWING

FIG. 1 represents the treatment process using digital block. Piecemeal removal of the overlying nail is similar.

FIG. 2 represents the course of nail change.

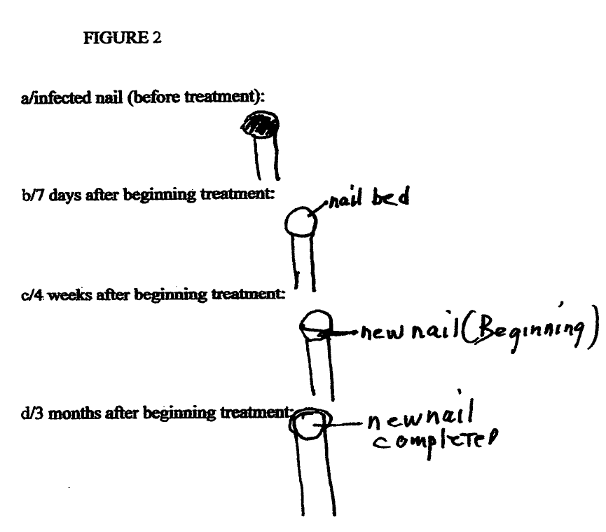

FIG. 3 represents possible usefull products/ accessories.

### DETAILED DESCRIPTION OF THE PREFERRED EMBODIMENTS OF INVENTION

Various aspects of the invention will evolve from the following detailed description of the preferred embodiments thereof, which should be referenced to the prior described drawings. This is especially expected to occur if this novel process is adopted to widespread use, and replaces existing oral and topical treatments. The essence of this invention is the process/products for curing onychomycosis of the nails of the hands and feet of all affected individuals.

What is claimed is:

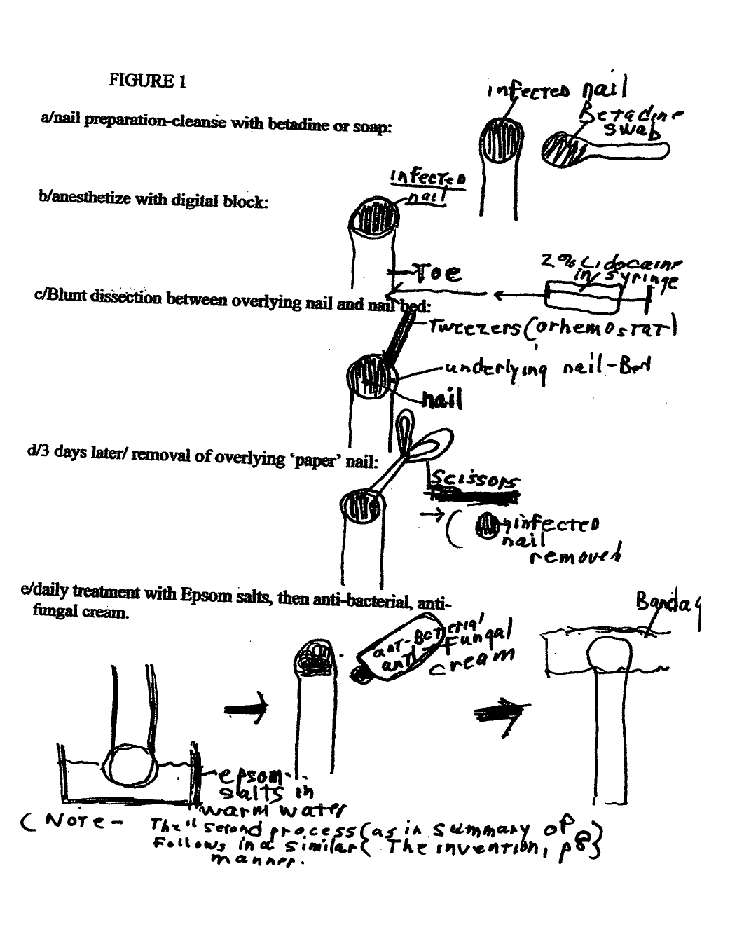

1/ The Invention, FIG. 1

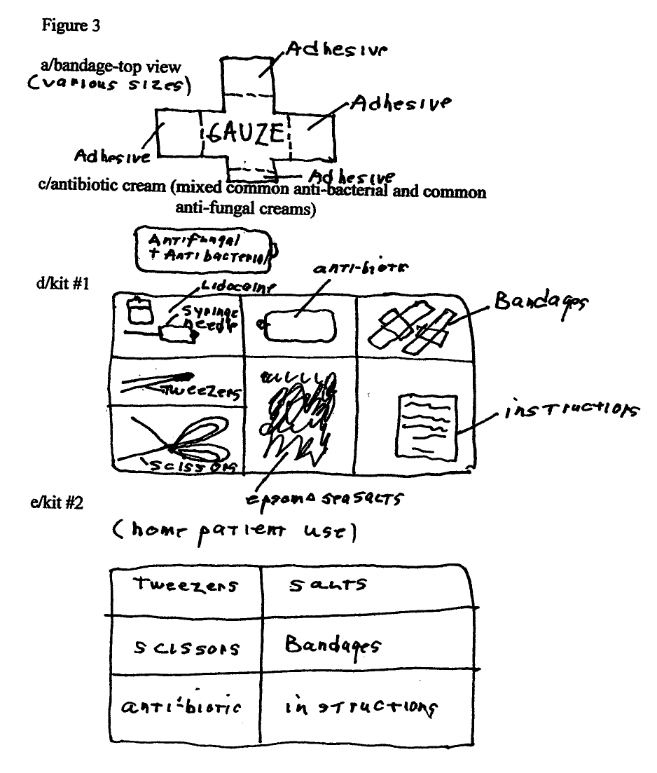

2/ Possible accessory devices to facilitate the use of the process. FIG. 3.
This could include:
a/new type of bandage to fit over the excised nail and contain the applied antibiotic.
c/anti-bacterial and anti-fungal components mixed into one cream, spray or ointment.
d/pre-packaged kit #1—(designed for physician office use)—includes directions, lidocaine, needle and syringe, tweezers or hemostat, scissors, anti-bacterial and anti-fungal cream, bandages.
e/pre-packaged kit # 2—(designed for home patient use)—includes directions, tweezers or hemostat, scissors, antibiotic cream or spray, bandages.
